(19) United States
(12) Patent Application Publication
(10) Pub. No.: US 2016/0066841 A1
(43) Pub. Date: Mar. 10, 2016

(54) STRAP BAND FOR A WEARABLE DEVICE

(71) Applicant: Sylvia Hou-Yan Cheng, San Francisco, CA (US)

(72) Inventor: Sylvia Hou-Yan Cheng, San Francisco, CA (US)

(73) Assignee: AliphCom, San Francisco, CA (US)

(21) Appl. No.: 14/480,048

(22) Filed: Sep. 8, 2014

(51) Int. Cl. A61B 5/00; A61B 5/0205

(52) U.S. Cl. CPC A61B 5/681; A61B 5/02055

(57) ABSTRACT

A strap band including a flexible wire bus having electrodes and wires coupled with the electrodes is described. The wire bus may be encapsulated in the strap band by a molding process. The wire bus may determine electrode positions relative to each other and other structure coupled with the strap band. The strap band may be coupled with a device that includes circuitry that drives signals on to some of the electrodes and receives signals from non-driven electrodes. The electrode spacing and strap band dimensions may be selected to form a strap band that may accommodate a wide range of user body sizes for a target region the electrodes are positioned in contact with.

### FIELD

Embodiments of the present application relate generally to hardware, software, wired and wireless communications, RF systems, wireless devices, wearable devices, biometric devices, health devices, fitness devices, and consumer electronic (CE) devices.

### BACKGROUND

Devices that may be used to detect and track motion, diet, sleep patterns, biometric data, fitness, and other activities of a user, must often be positioned on a user's body to sense signals or other data generated by the users body and/or motion of the user. In some applications, the device is worn on one of the bodies' extremities, such as the arm or wrist for example. Due to differences in size, shape and anatomy in a user base, some devices may require different sizes to accommodate those differences. For example, a wearable device may require small, medium and large sizes, or even an extra-large size to accommodate differences in user's bodies. Biometric and/or other types of sensors that may be included in the device may require consistent positioning and/or contact with portions of a user's body, such as the skin, for example. A band or strap used to connect the device with a user's body may be too stiff, uncomfortable to wear, or not easily adjusted to match the user's body. In some examples, data generated by sensors may be unreliable due to the device being too tightly coupled with the user's body. In other examples, when a device is too tight, it may cause sweating and moisture from that sweating may result in unreliable sensor data, as in the case when sensors are used for measuring skin conductivity (e.g., galvanic skin response). Tight coupling of the device to the user's body may also cause sensors that come into contact with the body to leave an imprint after the device has been removed. Finally, some devices may not be configured to collect biometric data when the user is in motion (e.g., during exercise) due to sensor movement relative to the user's body.

Accordingly, there is a need for apparatus and systems for devices that are adjustable to accommodate a wide range of anatomies in a single device size, are comfortable to wear, and accurately collect sensor data.

### BRIEF DESCRIPTION OF THE DRAWINGS

Various embodiments or examples (“examples”) are disclosed in the following detailed description and the accompanying drawings:

FIG. 1 depicts examples of a strap band positioned on a body portion;

FIG. 2 depicts a side view of a strap band coupled with a device;

FIG. 3 depicts a top plan view and a side view of a strap band;

FIG. 4 depicts profile views of a system including a strap band;

FIG. 5 depicts views of a strap band and relative dimensions and positions of components of the strap band;

FIG. 6 depicts a side view and top plan view of a wire bus;

FIG. 7 depicts various examples of electrodes;

FIG. 8 depicts examples of circuitry coupled with electrodes of a strap band; and

FIG. 9 depicts profile views of a systems that include a strap band

Although the above-described drawings depict various examples of the invention, the invention is not limited by the depicted examples. It is to be understood that, in the drawings, like reference numerals designate like structural elements. Also, it is understood that the drawings are not necessarily to scale.

### DETAILED DESCRIPTION

Various embodiments or examples may be implemented in numerous ways, including but not limited to implementation as a device, a wireless device, a system, a process, a method, an apparatus, a user interface, or a series of executable program instructions included on a non-transitory computer readable medium. Such as a non-transitory computer readable medium or a computer network where the program instructions are sent over optical, electronic, or wireless communication links and stored or otherwise fixed in a non-transitory computer readable medium. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

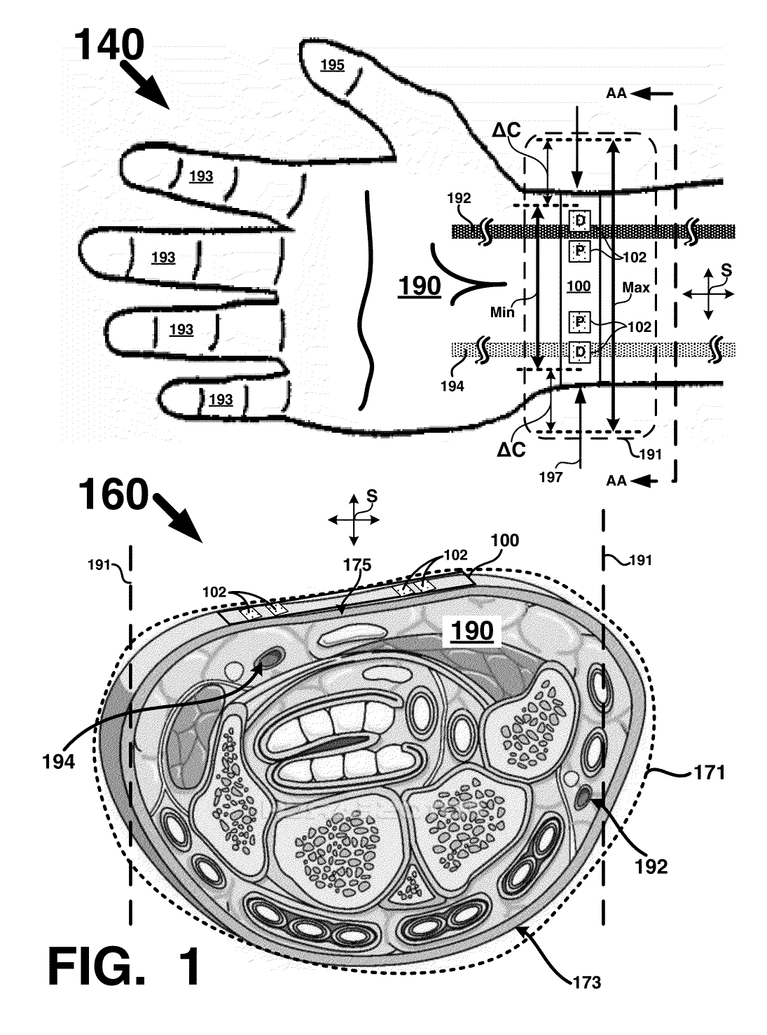

Reference is now made to FIG. 1 where examples 140 and 160 of a strap band 100 positioned on a body portion 190 are depicted. Here, for purposes of explanation, a non-limiting example of a body portion is a wrist; however, the present application is not limited to a wrist and strap band 100 may be used with other body portions, including but not limited to the torso, the neck, the head, the arm, the leg, and the ankle, for example.

In example 140, electrodes 102 of strap band 100 may be configured to sense signals, such as biometric signals, from structures of body portion 190 positioned in a target region 191. As one non-limiting example, the structure of interest may include the radial artery 192 and the ulnar artery 194. The radial artery 192 is the largest artery that traverses the front of the wrist and is positioned closest to thumb 195. Ulnar artery 194 runs along the ulnar nerve (not shown) and is positioned closest to the pinky finger 193. The radial 192 and ulnar arteries arch together in the palm of the hand and supply the fingers 193, thumb 195 and front of the hand with blood. A heart pulse rate may be detected by blood flow through the radial 192 and ulnar arteries, and particularly from the radial artery 192. Accordingly, strap band 100 and electrodes 102 may be positioned within the target region 191 to detect biometric signals associated with the body, such as heart rate, respiration rate, activity in the sympathetic nervous system (SNS) or other biometric data, for example.

Target region 191 is depicted as being wider than the wrist 190 and spanning a depth along the wrist 190 to illustrate that variations in body anatomy among a population of users will result in differences in wrist sizes and some user's may position the strap band 100 closer to the hand; whereas, other user's may position the strap band 100 further back from the hand. Now the view in example 140 is a ventral view of the hand 190; however, the wrist 190 has a circumference C that may vary ΔC among users. Arrows 194 indicate a width of the wrist 190 for the example 140; however, in a population of users, circumference (see 171 of example 160) of a wrist may vary from a minimum Min (e.g., a very small wrist) to a maximum Max (e.g., a very large wrist). To accommodate variations in wrist circumference ΔC from Min to Max, dimensions of strap band 100, dimensions of electrodes 102 and positions of the electrodes 102 relative to each other and relative to other structures the strap band 100 may be coupled with, may be selected to position the electrodes 102 within the target region 190 for wrist sizes spanning a minimum wrist size of about 135 mm in circumference to a maximum wrist size of about 180 mm in circumference, for example. In other examples, the dimensions and positions may be selected to position the electrodes 102 within the target region 190 for wrist sizes spanning a minimum wrist size of about 130 mm in circumference to a maximum wrist size of about 200 mm in circumference. For example, within the target region 190, electrodes of strap band 100 may be positioned to sense signals from the radial 192 and ulnar 194 arteries for wrist circumferences within the aforementioned 130 mm to 200 mm range, even when the strap band 100 overlays a flat or curved surface of the wrist 190 or is displaced to the left, the right, up, or down as denoted by arrow for S on wrist 190 due to variations in where user's like to place their strap bands on their wrist 190. Therefore, the strap band 100 may not require an exact centered location on writs 190 in order for electrodes 102 to sense signals from structure in the target region 191 (e.g., 192 and 194).

Some of the electrodes 102 may have signals applied to them (e.g., are driven) and are denoted as D; whereas, other electrodes 102 may pick up signals (e.g., receive signals) and are denoted as P. Positioning and sizing of the electrodes 102 that are adjacent to each other (e.g., a driven D electrode next to a pick-up P electrode) may be selected to prevent those electrodes from contacting each other when the strap band 100 is bent or otherwise curved when donned by the user. For example, if electrodes 102 lie on an approximately flat portion of wrist 190, then adjacent electrodes 102 (e.g., a D and P) may not be significantly urged inward toward each other because they are lying on an approximately planar surface. On the other hand, if electrodes 102 lie on a curved portion of wrist 190, then adjacent electrodes 102 (e.g., a D and P) may be urged inward toward each other, and if the adjacent electrodes are spaced to close to each other, then their inward deflection might bring them into contact with each other (e.g., they become electrically coupled) and the signal being received by the pick-up P electrode will be the signal being driven on the drive D electrode and not the signal from structure in target region 191.

Example 160 depicts a cross-sectional view of wrist 190 along a dashed line AA-AA. A circumference of the wrist 190 is denoted as 171 and will vary based on wrist size. As depicted, strap band 100 is positioned on a ventral portion of wrist 190 in a region 175 that is relatively flat; however, in the target region 191, moving left or right away from 175 towards the boundary of the target region 191, the surface of wrist 190 becomes curved. Moreover, wrist 190 has curvature in a region 173 of a dorsal portion of the wrist 190. Although many users will likely wear a device that includes the strap band 100 in a prescribed manner in which the electrodes 102 of the strap band 100 are placed against the bottom of the wrist 190 (e.g., the ventral portion), some users may prefer to place the strap band 100 and its electrodes 102 on the dorsal portion 173 where the surface of wrist 190 includes curvature. In either case, strap band dimensions and electrode dimensions and placement may be selected to establish sufficient contact of the electrodes 102 with skin of the wrist 190 within the target region 191 so that signals driven onto drive D electrodes are coupled with wrist 190 and signals from wrist 190 are received by pick-up electrodes P.

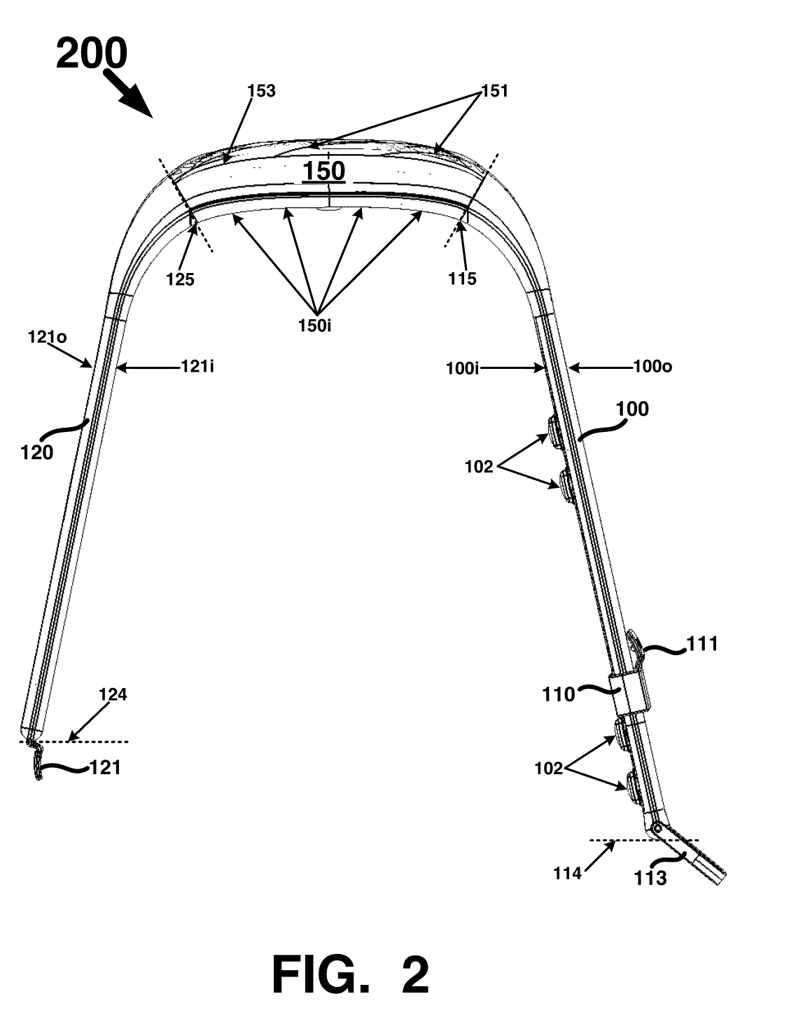

Moving now to FIG. 2 where a side view of a strap band 100 coupled with a device 150 is depicted. Here, device 150, a band 120, and strap band 100 may form a system 200. Device 150 may include circuitry, one or more processors (e.g., DSP, μP, μC), memory (e.g., non-volatile memory), data storage (e.g., for algorithms configured to execute on the one or more processors), one or more sensors (e.g., temperature, motion, biometric, ambient light), one or more radios (e.g., Bluetooth—BT, WiFi, near field communications—NFC), circuit boards, a power source, a display (e.g., LED, OLED, LCD), transducers (e.g., a loudspeaker, a microphone, a vibration engine), one or more antennas, a communications interface (e.g., USB), a capacitive touch interface, etc. for example. Device 150 may include an arcuate inner surface 150i having a curvature selected to prevent or minimize rotation of system 200 around wrist 190 (or other body portion) when system 200 is donned by a user. Preventing or minimizing rotation of system 200 may be operative to maintain position of electrodes 102 within the target region 191 and/or maintain contact between the electrodes 102 and skin within the target region 191. Device 150 may include ornamentation 151 (e.g., for esthetic purposes) on an upper surface 153.

Band 120 may be a mechanical band, that is, a band configured to couple with strap band 100 for donning system 200 on a body portion of a user, such as the wrist 190 of FIG. 1. Band 100 may be purely passive (e.g., no electronics disposed in it) or may be active (e.g., includes circuitry and/or passive and/or active electronic components). Band 120 may include a latch 121 configured to mechanically couple with a buckle 110 disposed on strap band 100. Latch 121 and a portion of band 120 may be inserted through a loop 113 disposed on strap band 100. Band 120 may include an inner surface 120i and an outer surface 120o. When band 120 is inserted into loop 113 and buckle 110 a portion of inner surface 120i may contact a portion of an outer surface 100o of strap band 100.

Strap band 100 may include a plurality of electrode 102 positioned on and extending outward of an inner surface 100i. Electrodes 102 and a portion of inner surface 100i may be positioned in contact with skin in target region 191 (e.g., skin on wrist 190) when the system 200 is donned by a user. In addition to electrodes 102, strap band 100 may house other components, such as wires for coupling electrodes 102 with circuitry, antenna, a power source, circuitry, integrated circuits (IC's), passive electronic components, active electronic components, etc., for example.

Strap band 100 and band 120 may couple with device 150 at attachment points denoted as 115 and 125 respectively. For purposes of explanation, attachment points 115 and 125 may be used as non-limiting examples of reference points for dimensions described herein. Further, dashed line 114 on strap band 100 and dashed line 124 on band 120 may be used as non-limiting examples of reference points for dimensions described herein.

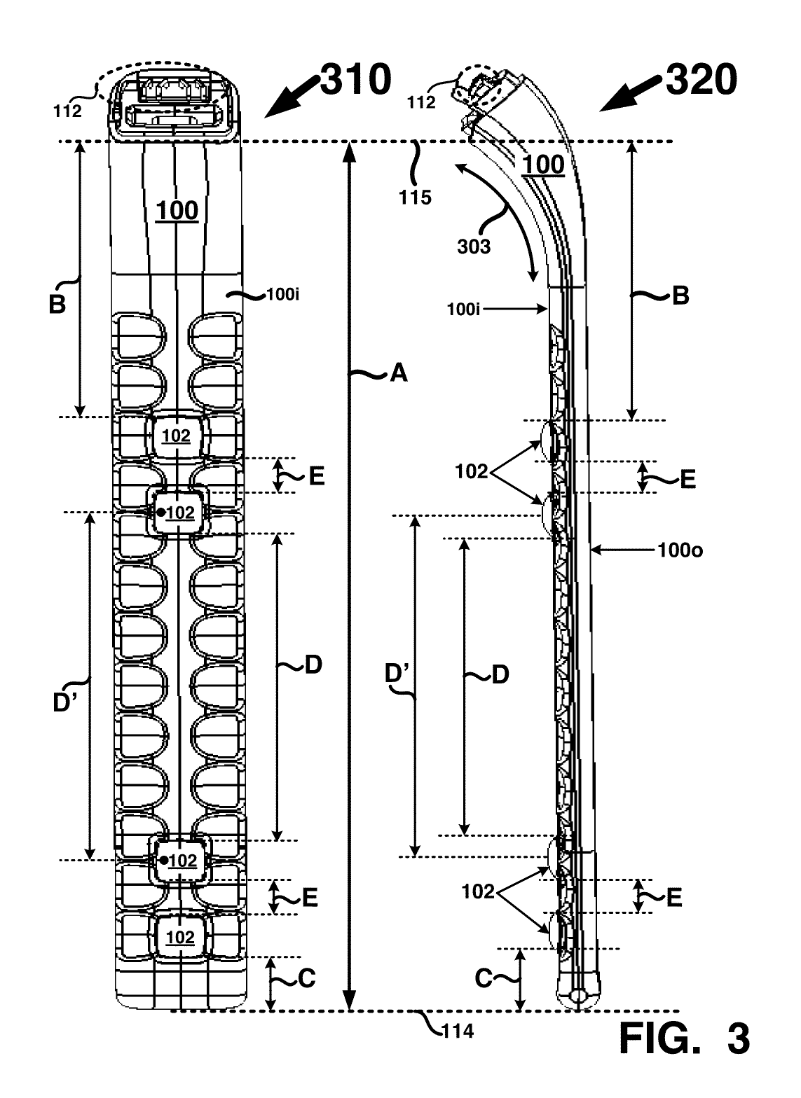

Turning now to FIG. 3 where a top plan view 310 and a side view 320 of a strap band 100 are depicted. In view 310 (e.g., looking down on inner surface 100i), dashed line 115 may serve as a reference point for dimensions A—E. Strap band 100 may include wires 112 that exit strap band 100 proximate its connection point with another structure, such as device 150 of FIG. 2, for example. Wires 112 may be coupled with electrodes 102 and may be coupled with circuitry (e.g., circuitry in device 150). An overall length of strap band 100 as measured from line 115 to line 114 may be dimension A. Dimension B may be a distance from line 115 to an edge of electrode 102. Dimension C may be a distance from line 114 to an edge of electrode 102. Dimension D may be a distance between inner facing edges of the two innermost electrodes 102. Dimension D′ may be a distance between centers of the two innermost electrodes 102, with distance D′ being greater than the distance D (i.e., D′>D). Dimension E may be a distance between edges of adjacent electrodes 102.

Dimensions A-E are presented in side view in view 320. In side view 320, strap band 100 may include an arcuate portion as denoted by arrows for 303. Strap band 100 may be flexible along its length (e.g., from 115 to 114). Although some dimensions other than D′ are measured from edge-to-edge (e.g., dimension E between edges of adjacent electrodes 102), center-to-center dimensions may also be used and the present application is not limited to edge-to-edge or center-to-center dimensions for measurements described herein. Side view 320 depicts electrodes 102 extending outward of inner surface 100i of strap band 100.

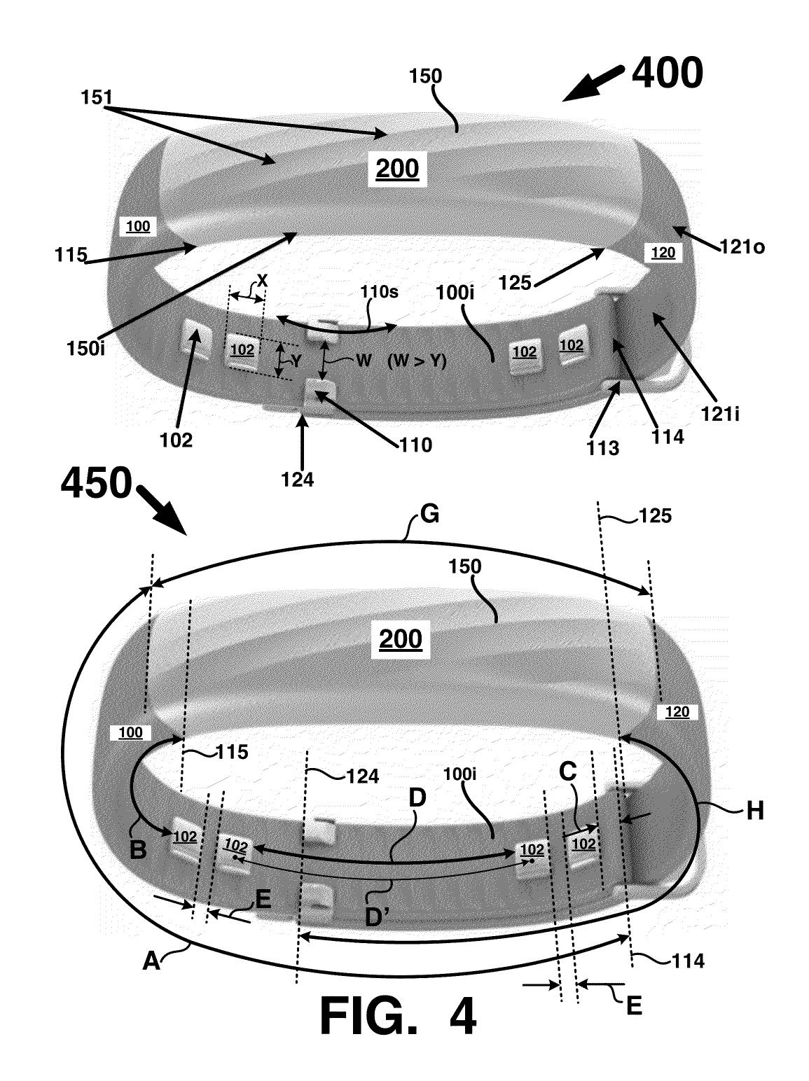

FIG. 4 depicts profile views 400 and 450 of a system 200 including strap band 100. Views 400 and 450 depict the system 200 in a configuration the system would have if donned on a user (e.g., system 200 attached to wrist 190 of FIG. 1). In view 400, device 150 is coupled with band 120 and strap band 100 with band 120 inserted through loop 113 and latch 121 coupled with buckle 110. Electrodes 102 are depicted positioned along inner surface 100i and having dimensions X and Y. Buckle 110 includes a gap having a width dimension W that is greater than the Y dimension of electrodes 102 (e.g., W>Y), so that sliding 110s buckle 110 along the strap band 100 in the direction of arrows for 110s will allow the buckle 110 to slide past the electrodes 102 without making contact with and without establishing electrical continuity with the electrodes 102.

Moving to view 450 where the aforementioned dimensions A-E are depicted along with dimensions for other components of system 200, namely, dimension G for device 150 and dimension H for band 120. Dimensions A-E, X, Y, W and G-H may be selected to form a system 200 that when donned by a user having a body portion circumference (e.g., a circumference of a wrist) in a range from about 130 mm to about 200 mm, will position the electrodes 102 within the target region 191 with sufficient contact force with skin in the target region to obtain a high signal-to-noise-ratio for circuitry that receives signals from pick-up electrodes P (e.g., the two innermost electrodes 102) in response from signals driven onto drive electrodes 102 (e.g., the two outermost electrodes 102). Although a range from about 135 mm to about 180 mm may be a typical range of wrist sizes found in a population of users, the larger range of from about 130 mm to about 200 mm may represent outlier ranges that are not typical but nevertheless may occasionally be encountered in a population of users. For example, a very skinny wrist of about 130 mm or a very large wrist of about 200 mm may be corner case exceptions to the more typical range beginning at about 135 mm and ending at about 180 mm of circumference.

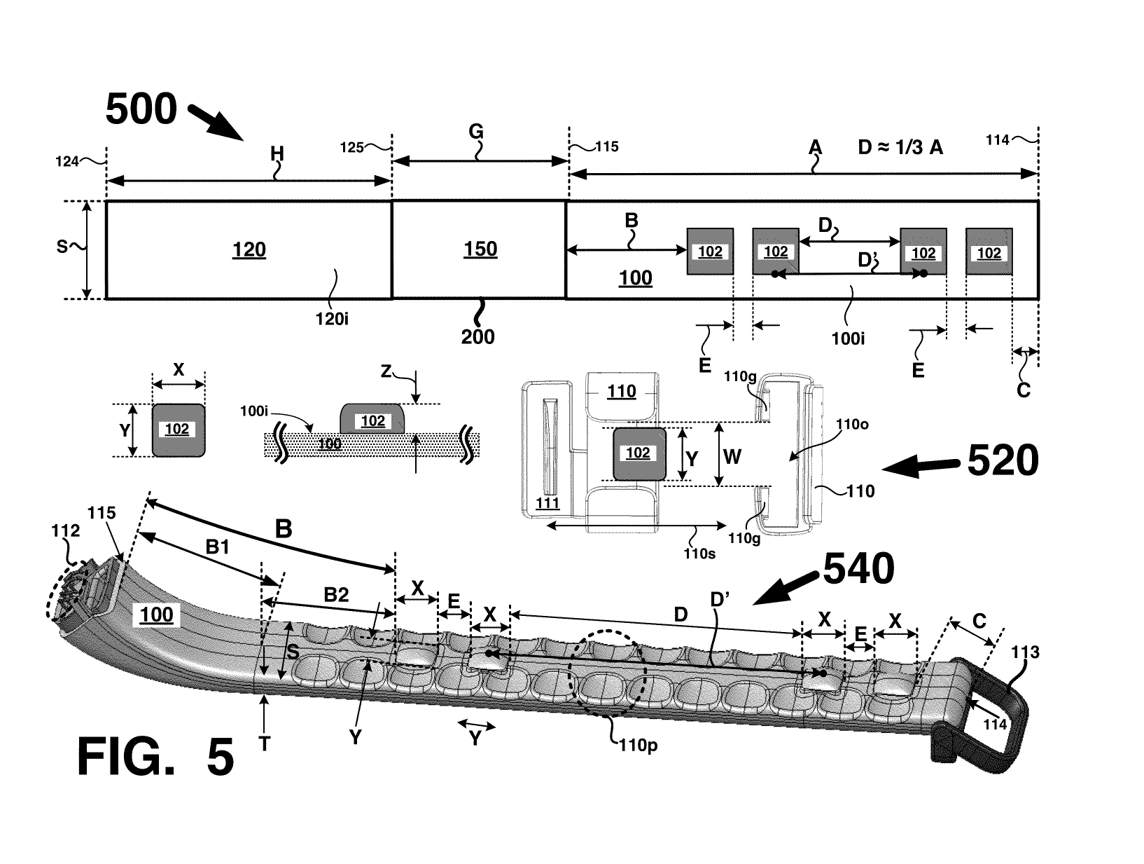

Reference is now made to FIG. 5 where views of strap band 100 and relative dimensions and positions of components of strap band 100 are depicted. In view 500, a system 200 may include the following example dimensions in millimeters (mm) with an example dimensional tolerance of +/−0.2 mm or less (e.g., +/−0.1 mm): dimension H for band 120 may be 80.0 mm (e.g., from 124 to 125 in FIG. 2); dimension G for device 150 may be 45.0 mm (e.g., from 125 to 115 in FIG. 2); dimension A for strap band 100 may be 95.0 mm (e.g., from 115 to 114 in FIG. 2); dimension B from 115 to an edge of outermost electrode 102 may be 32.0 mm; dimension E from an edge of outermost electrode 102 to an edge of adjacent innermost electrode 102 may be 4.0 mm; dimension D from an edge of innermost electrode 102 to an edge of the other innermost electrode 102 may be 31.5 mm edge-to-edge or dimension D′ for innermost electrodes 102 may be 36.0 mm center-to-center; distance E from an edge of innermost electrode 102 to the other outermost electrode 102 may be 4.0 mm; distance C from an edge of the outermost electrode 102 to 114 may be 5.5 mm; and a distance S of band 120, strap band 100 or both may be 10 mm-11 mm (e.g., a width of the band 120 and/or strap band 100). As one example, distance D may be approximately one-third (⅓) the dimension A for strap band 100, such that if A=95.0 mm, then D may be approximately 31.6 mm, with a tolerance of +/−0.2 mm or less (e.g., +/−0.1 mm).

In view 520, example dimensions for electrodes 102 may include a X dimension of 4.5 mm and a Y dimension of 4.5 mm. Electrodes 102 may have a height Z above inner surface 100i of strap band 100 of 1.5 mm. Dimensional tolerances for dimensions X, Y, and Z may be +/−0.2 mm or less (e.g., +/−0.1 mm). In view 520 dimension W of buckle 110 may be selected to be greater than dimension Y of electrode 102 to provide clearance between opposing edges of electrode 102 and buckle 110 so that as buckle 110 slides 110s along strap band 100, the buckle 110 does not make contact with electrodes 102 (e.g., the opposing edges). Dimension W may be selected to be about 0.3 mm to about 0.6 mm greater than dimension Y of electrodes 102. For example, if dimension Y is 4.5 mm, then dimension W may be 5.0 mm. Buckle 110 may include guides 110g configured to engage with features 110p on inner surface 100i of strap band 100 (see view 540). For example, prior to attaching loop 113 to strap band 100, strap band 100 may be inserted through an opening 110o of buckle 110 and guides 110g may engage features 110p to allow indexing (e.g., a mechanical stop) of the buckle 110 as it slides 110s along the strap band 100. The indexing may allow a user of the system 200 to adjust the fit of the system 200 to their individual wrist size (e.g., by sliding 110s the buckle 110 along strap band 100), while also providing tactile feedback caused by guides 110g engaging features 110p as the buckle slides 110s along the strap band 100. Guides 110g may also be operative to fix the position of the buckle 110 on the strap band 100 after the user adjustment has been made so that the buckle 110 does not move (e.g., buckle 100 remains stationary unless moved by the user).

Dimensions X, Y, and Z of electrodes 102 may be selected to determine a surface area of the electrodes 102 (e.g., for surfaces of electrodes 102 that are urged into contact with skin in target region 191). For example, surface area for electrodes 102 may be in a range from about 10 mm² to about 20 mm². In some examples, structure connected with the electrodes 102 may cover some portion of the surface of the electrodes 102 and/or sidewall surfaces of the electrodes 102 and reduce their actual surface area (e.g., skirts 104 that surround the electrodes 102, material of strap band 100). For example, with dimensions X and Y being 4.5 mm such that electrodes 102 have an actual surface area of 20.25 mm², an effective surface area of the electrodes 102 that may be exposed above inner surface 100i for contact with skin may be 18 mm².

In view 540, structure on inner surface 100i of strap band 100 is depicted in greater detail than in view 500. For example, proximate 115 a portion of dimension B may be arcuate and dimension B may include dimensions B1 and B2, where dimension B1 may be the curved portion of B. The Y dimension for only one of the electrodes 102 is depicted; however, for purposes of explanation it may be assumed that the Y dimensions of the other electrodes 102 are identical. In view 540, strap band 100 may have a width S of 10.0 mm and a thickness T of 2.0 mm measured between inner 100i and outer 100o surfaces. Thickness T may be the thinnest section of strap band 100 and strap band 100 may be thicker along portions of dimension B1. Thickness T may be in a range from about 0.9 mm to about 3.2 mm, for example. The following are another example of dimensions in millimeters (mm) for strap band 100 with example dimensional tolerances of +/−0.2 mm or less (e.g., +/−0.1 mm): dimension B1 may be 16.91 mm; dimension B2 may be 15.02 mm; dimension X for electrodes 102 may be 4.46 mm; dimension Y for electrodes 102 may be 4.46 mm; dimension E between adjacent electrodes 102 may be 3.54 mm; may be 3.54 mm; dimension D (edge-to-edge) may be 32.54 mm or D′ (center-to-center) may be 37.0 mm; and distance C may be 5.96 mm.

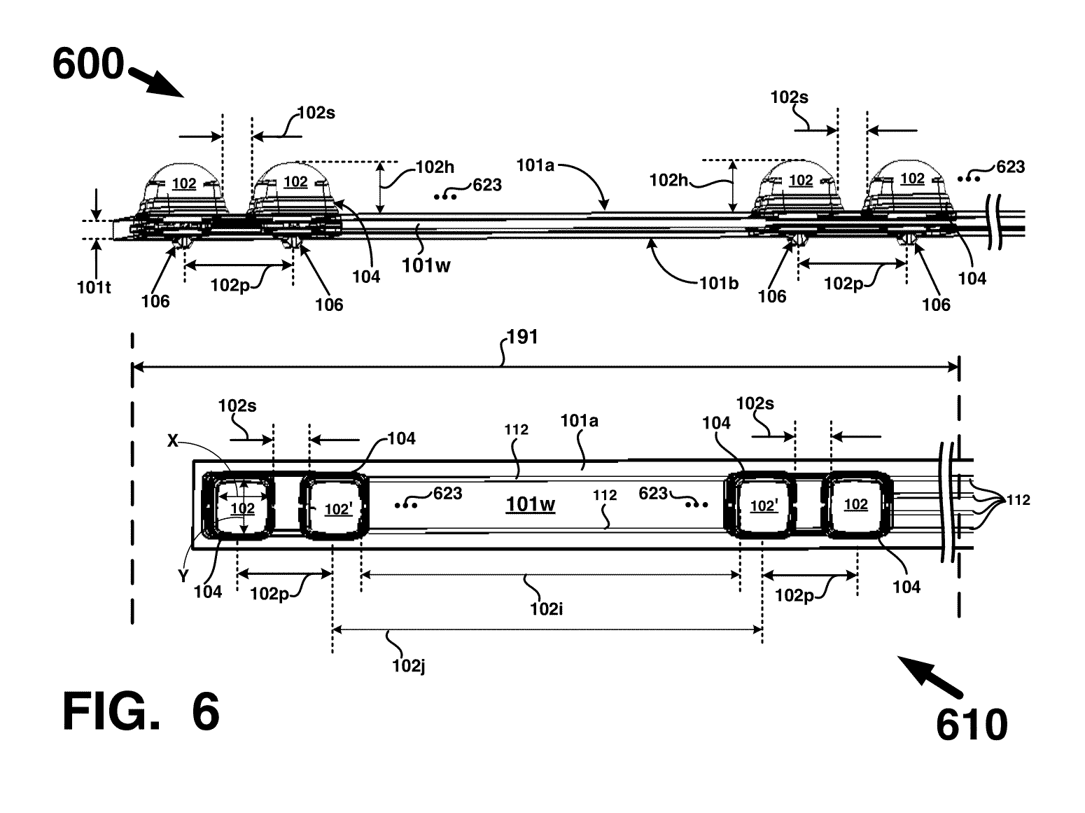

Attention is now directed to FIG. 6 where side view 600 and top plan view 610 of a wire bus 101w is depicted. Wire bus 101w may be a sub-assembly that is encapsulated (e.g., by injection molding) or otherwise incorporated into strap band 100. Electrodes 102 may be mounted on wire bus 101w and wires 112 may be connected with electrodes 102 by a process such as soldering, welding, crimping, for example. Some of the dimensions as described above in regards to FIGS. 3-5 may be determined in part by dimensions and placement of electrodes 102 on wire bus 101w. As one example a length of wire bus 101w may be selected to span dimension A of strap band 100 so that electrodes 102 on wire bus 101w are positioned within the target range 191. Similarly, dimensions B, E, X, Y, D, D′, C, S, and T on strap band 100 may be determined in part by dimensions, positions and sizes of electrodes 102 on wire bus 101w. Wire bus 101w may be made from a material such as a thermoplastic elastomer (e.g., TPE or TPU). The material for wire bus 101w may be a flexible material. Wire bus 101w may have a thickness 101t in a range from about 0.3 mm to about 1.1 mm, for example. Skirt 104 may be made from a polycarbonate material, for example.

Electrodes 102 may include pins 106 used in mounting the electrodes 102 to wire bus 101w. A distance (e.g., a pitch) between centers of pins 106 may determine the spacing between electrodes 102 on strap band 100. For example, spacing 106 may determine an edge-to-edge distance 102s between adjacent electrodes 102 and the distance 102s may determine distance E on strap band 100. As another example, an edge-to-edge distance 102i or a center-to-center distance 102j between the innermost electrodes 102′ may determine distances D and D′ respectively on strap band 100. A height 102h from a surface 101a of wire bus 101w to a top of electrodes 102 may determine height Z (see view 520 of FIG. 5) on strap band 100, for example. Due to the material used to form the strap band 100 over the wire bus 101w the dimension for Z will typically be less than the dimension for 102h. For example, if Z is 1.5 mm, then 102h may be 1.7 mm. There may be more or fewer electrodes 102 on wire bus 101w as denoted by 623. Skirts 104 may be coupled with electrodes 102 and may be operative as an interface between materials for the strap band 100 and electrodes 102 and may form a seal around the electrodes 102. Skirts 104 and material used to form the strap band 100 around the wire bus 101w may reduce actual surface area of the electrodes to an effective surface area as described above.

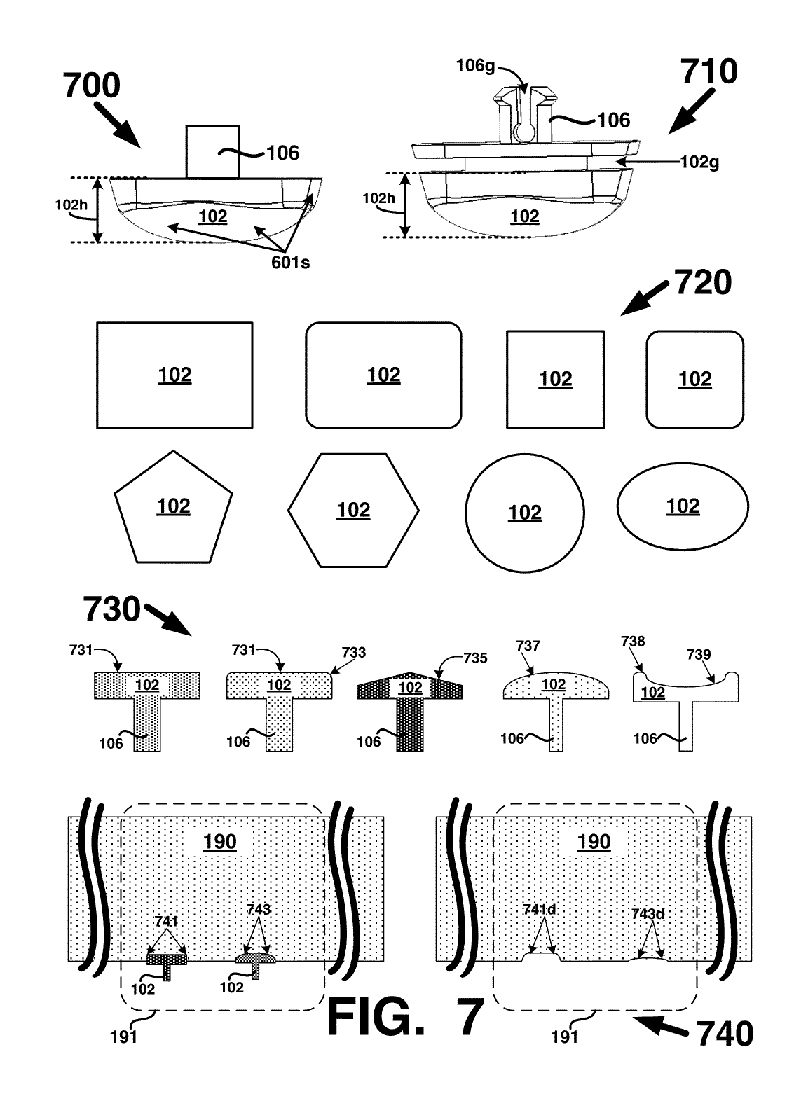

FIG. 7 depicts various examples of electrodes 102. In example 700, electrode 102 may include an arcuate surface and a pin 106. Height 102h may be measured from a top surface to a bottom surface of electrode 102. In example 710, electrode 102 may include a groove 102g and a pin 106 that includes a slot 106g. Height 102h may be measured from a top surface to a surface of groove 102g. Groove 102g may be surrounded by skirt 104 described above in reference to FIG. 6.

In example 720, different shaped for electrode 102 are depicted. Electrode 102 may have a shape including but not limited to a rectangular shape, a rectangle with rounded corners, a square shape, a square with rounded corners, a pentagon shape, a hexagon shape, a circular shape, and an oval shape, for example.

In example 730, surfaces of electrode 102 may have surface profiles including but not limited to a planar surface 731, a planar surface 731 with rounded edges 733, a sloped surface 735, an arcuate surface 737 (e.g., convex), and an arcuate surface 739 (e.g., concave). Arcuate surface 739 may include rounded edges 738. Surface profiles of electrodes 102 may be configured to maximize surface area of the electrodes 102 that contact skin, to provide a comfortable interface between the electrode and the user's skin (e.g., for prolong periods of use, such as 24/7 use), to maximize electrical conductivity for improved signal to noise ratio (S/N), for example.

In example 740, electrode 102 with a planar surface profile 741 and electrode 102 having an arcuate surface profile 743 are depicted engaged with skin of body portion 190 (e.g., a wrist). After the electrodes 102 are disengaged with the skin, each electrode 102 may leave an impression in the skin denoted as 741d and 743d. After a period of time has elapsed after the disengaging, the impression 743d from the electrode 102 having the arcuate surface profile 743 may be less pronounced and may fade away faster than the more pronounce impression 741d left by the electrode 102 with the planar surface profile 741. Accordingly, some surface profiles for electrodes 102 may be more desirable for esthetic purposes (e.g., minimal impression after removal) and for comfort purposes (e.g., sharp edges may be uncomfortable).

Suitable materials for electrodes 102 include but are not limited to metal, metal alloys, stainless steel, titanium, silver, gold, platinum, and electrically conductive composite materials, for example. Electrodes 102 may be coated 601s with a material operative to improve signal capture, such as silver or silver chloride, for example. Electrodes 102 may be coated 601s with a material operative to prevent corrosion or other chemical reactions that may reduce electrical conductivity of the electrodes 102 are damage the material of the electrodes 102. Examples of substances that may cause corrosion or other chemical reactions include but are not limited to body fluids such as sweat or tears, salt water, chlorine (e.g., from swimming pools), water, household cleaning fluids, etc.

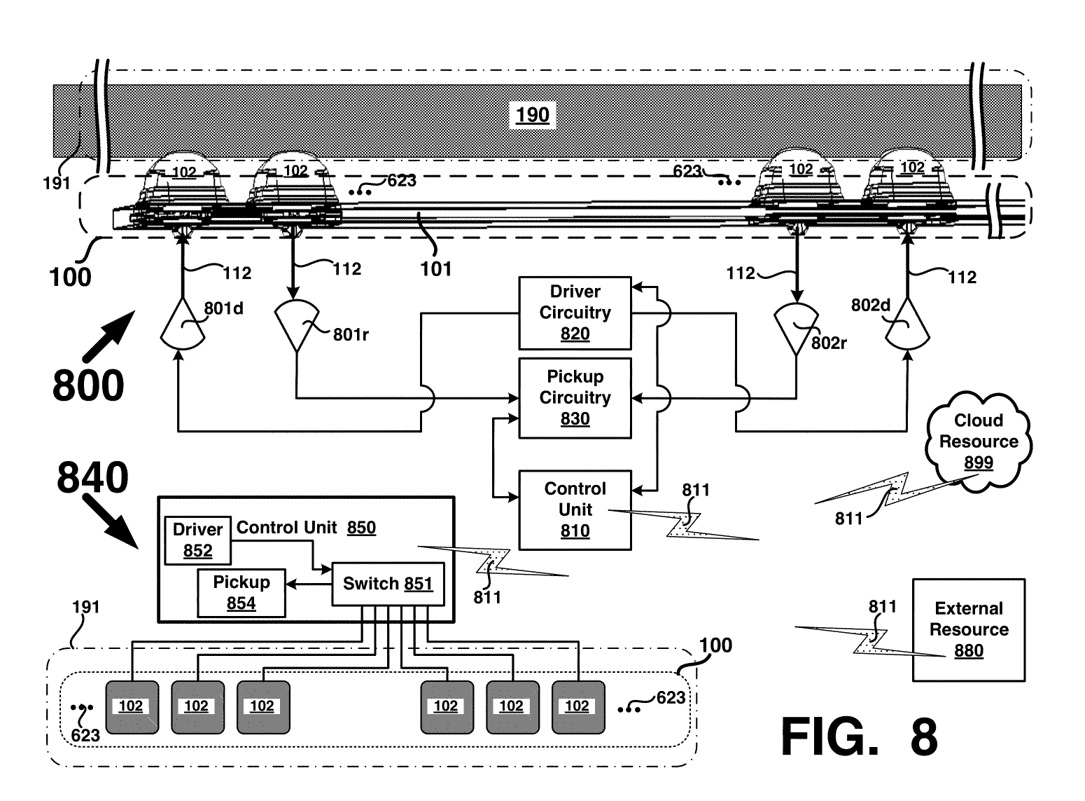

Reference is now made to FIG. 8 where examples of circuitry coupled with electrodes 102 of a strap band 100 are depicted. In example 800, electrodes 102 are depicted engaged into contact with skin of body portion 190 within target region 191. Outermost electrodes 102 may be coupled (e.g., via wires 112) with drivers 801d and 802d operative to apply a signal to the outermost electrodes 102 (e.g., driven D electrodes 102). Innermost electrodes 102 may be coupled (e.g., via wires 112) with receivers 801r and 802r operative to receive signals picked up by innermost electrodes 102 from electrical activity on the surface of and/or within body portion 190. Drivers 801d and 802d may be coupled with driver circuitry 820 and receivers 801r and 802r may be coupled with pickup circuitry 830. A control unit 810 may be coupled with driver circuitry 820 and with pickup circuitry 830. Control unit 810 may include one or more processors, data storage, memory, and algorithms operative to control driver circuitry 820 and pickup circuitry 830 to process data received by pickup circuitry 830, and to generate data used by driver circuitry 820 to output driver signals coupled with drivers 801d and 802d, for example. As one example, electrodes 102 may sense and/or generate signals associated with biometric functions of the body, such as bioimpedance (BI). Control unit 810 may perform signal processing of signals associated with driver circuitry 820 and/or pickup circuitry 830, or an external resource 880 and/or cloud resource 899 in communication 811 (e.g., via a wired or wireless communication link) may perform some or all of the processing. For example, control unit 810 may transmit 811 data to 880 and/or 899 for processing. External resource 880 and/or cloud resource 899 may include or have access to compute engines, data storage, and algorithms that are used to perform the processing.

In example 840, strap band 100 may include a plurality of electrodes 102 coupled with a switch 851 that is controlled by a control unit 850. Control unit 850 may command switch 851 to couple one or more of the electrodes 102 with driver circuitry 852 such that electrodes 102 so coupled become driven electrodes D. Control unit 850 may command switch 851 to couple one or more of other electrodes 102 with pickup circuitry 854 such that electrodes 102 so coupled become pick-up electrodes P. There may be more or fewer of the electrodes 102 as denoted by 623. Processing of signals and/or data may be handled by control unit 850 and/or by external resource 880 and/or cloud resource 899 using communications link 811 as described above. Algorithms and/or data used in the processing may be embodied in a non-transitory computer readable medium (e.g., non-volatile memory, disk drive, solid state drive, DRAM, ROM, SRAM, Flash memory, etc.) configured to execute on one or more processors, compute engines or other compute resources in control unit 810, 850, external resource 880 and cloud resource 899. Electrodes 102 in example 840 may be used to cover additional surface area on body portion 190 as may be needed to accommodate differences in size of body portion 190 among a user population. External resource 880 may be a wireless client device, such as a smartphone, tablet, pad, PC or laptop and may execute an algorithm or application (APP) operative to determine which electrodes 102 to activate via switch 851 as driver D or pick-up P electrodes. A user may enter information about their wrist size or other body portion size as data used by the APP to make electrode 102 selections. Control unit 810 and/or 850 may be included in device 150 of FIG. 2, for example.

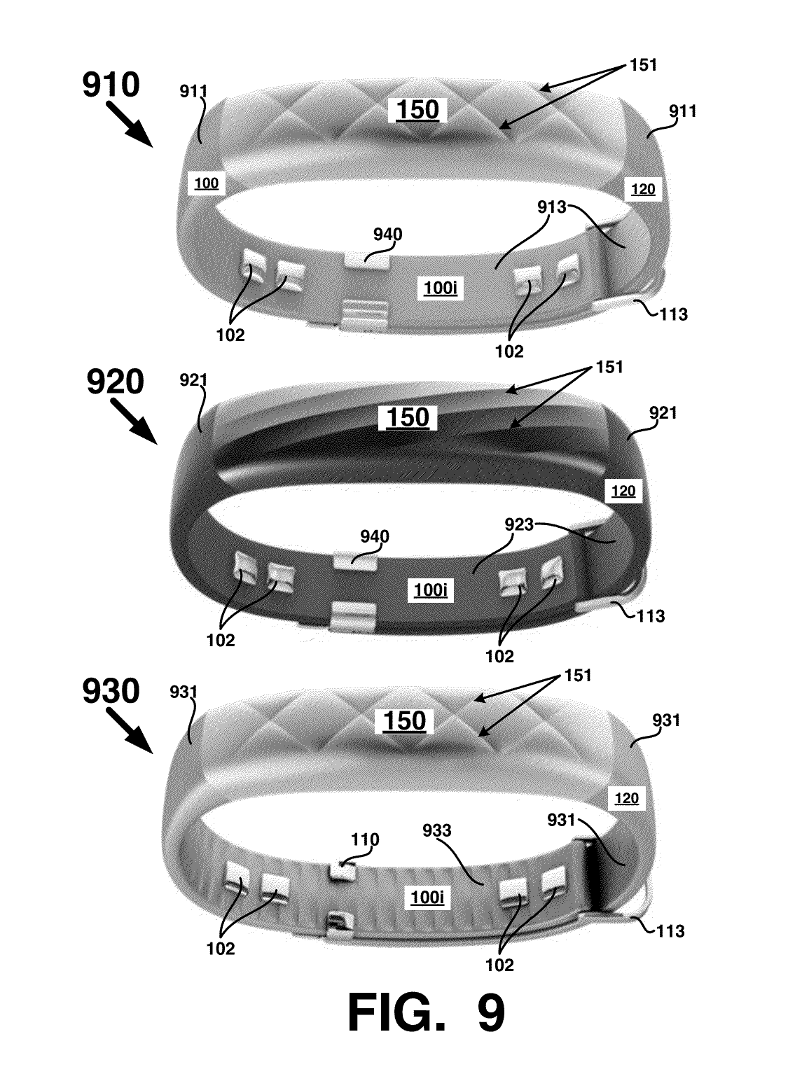

FIG. 9 depicts profile views of systems 910-930 that include strap band 100. System 910 may include device 150, band 120, and strap band 100. Band 120 and strap band 100 may be made from a thermoplastic elastomer such as TPE, TPU, TPSV, or others, for example. The thermoplastic elastomer may be covered with an exterior fabric material 911, such as cloth or nylon, for example. The electrode 102 and fastening hardware 113, 121, 940 may be anodized or coated with a surface finish such as a colored chrome finish, for example. In system 910, buckle 110 may be replaced with a buckle 940 configured to slide 110s along the exterior fabric material 911 without damaging the fabric material 911.

System 920 may include a faux leather exterior surface material 921 which may have a variety of finishes such as matte, flat, glossy, etc. The fastening hardware of system 920 may be coated with a surface finish as described above.

System 930 includes band 120 and strap band 100 that may be from a material 931, such as a thermoplastic elastomer such as TPE, TPU, TPSV, or others, for example. Inner surface 100i of strap band 100 includes features operative to index buckle 110 as was described above in reference to FIG. 5. Material 921 which may have a variety of finishes such as matte, flat, glossy, etc. The fastening hardware of system 930 may be coated with a surface finish as described above.

Device 150 may include top and bottom portions made from a material such as anodize aluminum that may be anodized in a variety of colors, for example. An upper surface may include ornamental elements 151.

Although the foregoing examples have been described in some detail for purposes of clarity of understanding, the above-described inventive techniques are not limited to the details provided. There are many alternative ways of implementing the above-described techniques or the present application. The disclosed examples are illustrative and not restrictive.

What is claimed is:

1. A system, comprising:
a strap band including an encapsulated wire bus having a plurality of electrodes connected with the wire bus, the wire bus including wires, each wire connected with one of the plurality of electrodes, an upper surface of each electrode is sealed by a material of the strap band and extends outward of an inner surface of the strap band;
a band;
fastening hardware coupled with the band and with the strap band; and
a device including circuitry coupled with the wires, the band and the strap band coupled to the device at opposing ends of the device.

2. The system of claim 1, wherein the fastening hardware includes a buckle including an electrode gap, the buckle operative to slide along the strap band, the electrode gap configured to have a width wider that a dimension of the plurality of electrodes so that the no portion of the buckle comes into contact with electrodes.

3. The system of claim 1, wherein the buckle is made from an electrically conductive material.

4. The system of claim 1, wherein each electrode includes an arcuate surface that extends outward of the inner surface by a distance of approximately 1.5 mm.

5. The system of claim 1, wherein the plurality of electrodes includes two pairs of adjacent electrodes, the adjacent electrodes in each pair are spaced apart from each other by a distance of approximately 4.0 mm.

6. The system of claim 5, wherein the distance comprises an edge-to-edge distance.

7. The system of claim 1, wherein the plurality of electrodes includes two pairs of adjacent electrodes, and innermost electrodes in each pair are spaced apart from each other by a distance of approximately 31.5 mm.

8. The system of claim 7, wherein the distance comprises an edge-to-edge distance.

9. The system of claim 1, wherein the plurality of electrodes includes two pairs of adjacent electrodes, and innermost electrodes in each pair are spaced apart from each other by a distance of approximately 36.0 mm.

10. The system of claim 7, wherein the distance comprises a center-to-center distance.

11. The system of claim 1, wherein the circuitry electrically couples two or more of the plurality of electrodes to driver circuitry and couples another two or more of the plurality of electrodes to pickup circuitry.

12. The system of claim 1, wherein the plurality of electrodes includes two pairs of adjacent electrodes, a first outermost electrode in a first of the two pairs is positioned a distance of approximately 32 mm from the device, and a second outermost electrode in a second of the two pairs is positioned a distance of approximately 5.5 mm from a distal end of the strap band.

13. The system of claim 1, wherein the device include an arcuate inner surface.

14. The system of claim 1, wherein the strap band, the band or both are made from a thermoplastic elastomer material.

15. The system of claim 1, wherein each electrode has a square shape with dimensions of approximately 4.5 mm on a side.

16. An apparatus, comprising:
a strap band; and
a wire bus encapsulated in the strap band and including a plurality of electrodes, each electrode coupled with a wire, each wire routed to and exits out of a first end of the strap band, each electrode including an upper surface that extends outward of an inner surface of the strap band, the plurality of electrodes are grouped into two pairs with each pair including two electrodes that are adjacent to each other, adjacent electrodes in the two pairs are spaced apart from each other by an identical distance, and innermost electrodes in each pair are spaced apart by a distance that is approximately one-third of a length of the strap band.

17. The apparatus of claim 16, wherein the length is approximately 95.0 mm.

18. The apparatus of claim 16, wherein the innermost electrodes in each pair are spaced apart by a distance of approximately 31.5 mm.

19. The apparatus of claim 16, wherein the identical distance is approximately 4.0 mm.

20. The apparatus of claim 16, wherein an outermost electrode in a first of the two pairs is spaced apart from the first end by a distance of approximately 32.0 mm and another outermost electrode in a second of the two pairs is spaced apart from a second end of the strap band by a distance of approximately 5.5 mm.
